(19) United States
(12) Patent Application Publication
(10) Pub. No.: US 2007/0077200 A1
(43) Pub. Date: Apr. 5, 2007

(54) Method and system for controlled maintenance of hypoxia for therapeutic or diagnostic purposes

(76) Inventor: Clark R. Baker, Castro Valley, CA (US)

Correspondence Address: FLETCHER YODER (TYCO INTERNATIONAL, LTD.), P.O. BOX 692289, HOUSTON, TX, 77269-2289, US

(21) Appl. No.: 11/241,062

(22) Filed: Sep. 30, 2005

(51) Int. Cl. A61K 49/00; A62B 7/00; A61K 33/00

(52) U.S. Cl. 424/9.1; 424/600; 128/200.24

(57) ABSTRACT

Embodiments of the present invention relate to a system, device, and method for automatically inducing, maintaining, or controlling hypoxia in a patient. Specifically, embodiments of the present invention relate to delivering a hypoxic gas mixture to a patient, monitoring at least one physiological parameter of the patient, and automatically controlling the delivery of the hypoxic gas mixture based on a value of the physiological parameter.

### BACKGROUND OF THE INVENTION

1. Field of the Invention

The present invention relates generally to a method and system for inducing, maintaining, and/or controlling hypoxia in a patient by controlled delivery of a hypoxic gas mixture to the patient. Specifically, embodiments of the present invention are directed to closed-loop control of a delivery rate and/or composition of the hypoxic gas mixture being inhaled by the patient to facilitate safe inducement, maintenance, and/or control of patient hypoxia for diagnostic and/or therapeutic purposes.

2. Description of the Related Art

This section is intended to introduce the reader to various aspects of art that may be related to various aspects of the present invention, which are described and/or claimed below. This discussion is believed to be helpful in providing the reader with background information to facilitate a better understanding of the various aspects of the present invention. Accordingly, it should be understood that these statements are to be read in this light, and not as admissions of prior art.

Hypoxia, in contrast to normoxia (normal oxygen concentration) and anoxia (complete or near absence of oxygen), relates to a subnormal concentration of oxygen in a patient's blood. Hypoxia may be defined as a pathological condition in which the entire body or an area of the body is deprived of adequate oxygen supply. When the body as a whole is deprived of adequate oxygen supply, it may be referred to as generalized hypoxia. When a certain region of the body is deprived of adequate oxygen supply, it may be referred to as tissue or local hypoxia. Hypoxia, if severe enough, can cause tissue damage and even cell death.

In the vast majority of healthcare settings, hypoxia is a condition that should be minimized and avoided. However, patient hypoxia can be beneficial for some therapeutic and diagnostic measures. For example, in neonatal intensive care units (NICU), maintenance of limited hypoxia is often desirable because it can prevent retinopathy of prematurity (i.e., a disorder of the blood vessels of the retina that is common in premature babies). Additionally, there are several other conditions in which local hypoxia has diagnostic or therapeutic value. For example, tumors can be treated by repetitively inducing tumor hypoxia to kill tumor cells and achieve a desired degree of tumor remission. In some situations wherein a condition of hypoxia may be beneficial, manual inducement of hypoxia has been clinically accepted.

### BRIEF DESCRIPTION OF THE DRAWINGS

Advantages of the invention may become apparent upon reading the following detailed description and upon reference to the drawings in which:

FIG. 1 is a block diagram of a ventilation system that induces, maintains, or controls hypoxia in a patient in accordance with an exemplary embodiment of the present invention;

FIG. 2 is a graph illustrating data representative of automatically controlled hypoxia using an implementation of an exemplary embodiment of the present invention; and

FIG. 3 is a block diagram of a method illustrating an exemplary embodiment of the present invention.

### DETAILED DESCRIPTION OF SPECIFIC EMBODIMENTS

One or more specific embodiments of the present invention will be described below. In an effort to provide a concise description of these embodiments, not all features of an actual implementation are described in the specification. It should be appreciated that in the development of any such actual implementation, as in any engineering or design project, numerous implementation-specific decisions may be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

Embodiments of the present invention are directed to automated control of a composition and/or delivery amount of a hypoxic gas mixture to a patient to safely induce, maintain, and/or control hypoxia in the patient. Indeed, closed-loop control of a hypoxic gas mixture can be used to temporarily and safely increase a volume of hypoxic tissue, so as to maximize efficacy of a treatment, sensitivity of a diagnosis, and so forth. For example, automatic adjustment of FiO2 by a computer based controller, such as a proportional (P) controller, a proportional-integral (PI) controller, a proportional-integral-derivative (PID) controller, or a proportional-derivative (PD) controller may be utilized to control patient hypoxia, thus facilitating detection of tumors in the patient. It should be noted that FiO2 may be defined as the percentage of oxygen in air inhaled by a patient through a ventilator. For example, in typical room air, the value for FiO2 is approximately 21%.

Automated control of patient hypoxia may be beneficial to diagnostic or imaging procedures for detection of local hypoxia, such as tumor detection, detection of ischemic tissue (i.e., tissue having inadequate blood supply for its requirements of oxygen, nutrients, and removal of metabolic by-products), and delivery of an agent designed to localize in hypoxic tissue. Additionally, automated control of patient hypoxia may be utilized to create or enhance a therapeutic response dependent on local hypoxia. For example, such a therapeutic response may include neurogenesis (i.e., production of new nervous tissue) or apoptosis (i.e., programmed cell death or cellular suicide). Further, it should be noted that apoptosis created or enhanced in accordance with present embodiments may be mediated by providing an agent designed to localize in hypoxic tissue, by destruction of local vasculature, or by repetitive ischemia-reperfusion injury (i.e., inducement of cell damage via a bi-phasic process).

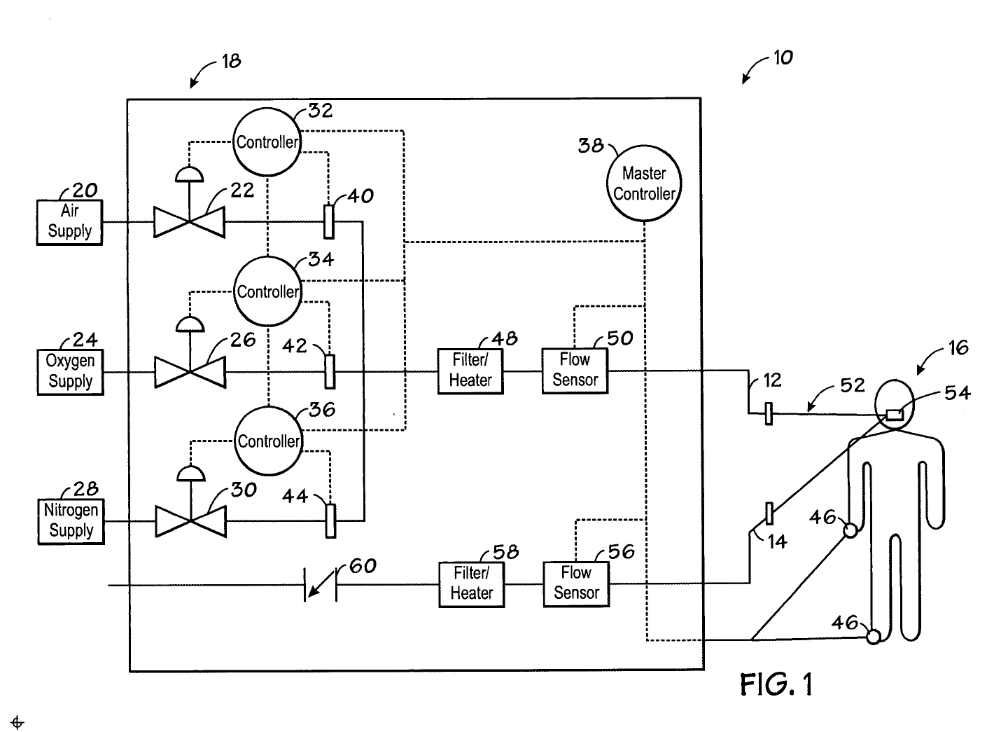

FIG. 1 is a block diagram of a ventilation system with a controllable hypoxic gas mixture supply mechanism and a controller for inducing, maintaining, and/or controlling patient hypoxia. The entire ventilation system is generally referred as ventilation system 10. In the illustrated embodiment, ventilation system 10 includes an inspiration line 12 and an expiration line 14. The inspiration line 12 provides a controlled gas mixture for a patient 16 to breath. The expiration line 14 receives gases (e.g., oxygen and carbon dioxide) exhaled by the patient 16. It should be noted that in some embodiments the ventilation system 10 includes an open exhalation line rather than the expiration line 14. In embodiments that implement the open exhalation line, gases exhaled by the patient do not pass back through the ventilation system 10 but simply pass directly into the atmosphere. Depending on application requirements, the open exhalation line or the expiration line 14 may be utilized to provide for safe operation or to facilitate certain procedures.

In the illustrated embodiment, an inlet portion 18 of the ventilation system 10 includes an air supply 20 coupled to an air valve 22, an oxygen supply 24 coupled to an oxygen valve 26, and a nitrogen supply 28 coupled to a nitrogen valve 30. The inlet portion 18 is designed to provide a defined gas mixture (e.g., a hypoxic gas mixture) to the inspiration line 12. The supplies 20, 24, and 28 and valves 22, 26, and 30 may be utilized to produce normal and hypoxic gas mixtures for supply to the patient 16. Inclusion of the oxygen supply 24 may be desirable in some situations wherein a rapid increase in FiO2 levels is desirable. However, it should be noted that some embodiments do not utilize the oxygen supply 24 but rely on the air supply for oxygen content in the normal or hypoxic gas mixture.

In the illustrated embodiment, each of the gas supplies 20, 24, and 28 may include a high pressure tank or cylinder with pressurized air, nitrogen, or oxygen disposed respectively therein. The valves 22, 26, and 30 and/or additional valves may operate to normalize the pressure and ensure desired gas mixture proportions. In one embodiment, the air supply 20 is the local atmosphere. That is, the air may be taken directly from the atmosphere using, for example, an air pump coupled to the air valve 22 in the inlet portion 10 of the ventilation system 10. Additionally, in some embodiments, a premixed hypoxic gas mixture supply is provided and regulated with a hypoxic gas mixture valve that facilitates combination with air or oxygen. The premixed hypoxic gas mixture may be supplemented with oxygen, air or both, and it may eliminate the need for the nitrogen supply 28.

Each of the valves 22, 26, and 28 in the inlet portion 18 of the ventilation system may be a control valve, such as an electronic, pneumatic, or hydraulic control valve, that is communicatively coupled to a controller (e.g., flow controller or differential pressure controller), as illustrated by controllers 32, 34, and 36, respectively. The controllers 32, 34, and 36 may receive a set point value from a master controller 38 that controls hypoxia in the patient 16. For example, each of the set points for the controllers 32, 34, and 36 may include a volume of flow for each particular type of gas (e.g., air, oxygen, and nitrogen). To maintain hypoxia, the master controller 38 may supply set points or predefined curves (e.g., hysteresis curves) to the controllers 32, 34, and 36 such that levels of FiO2 gradually fall to hypoxic levels from a normal starting gas supply composition. The controllers 32, 34, and 36 may monitor flow sensors 40, 42, and 44 and open or close the valves 22, 26, and 28 depending on the amount of flow of each type of gas. These adjustments may maintain or control gas compositions in the inspiration line 12, as designated by the set points and/or curves from the master controller 38.

The illustrated controllers 32, 34, 36, and 38 may each include an input circuit configured to receive real-world data (e.g., a monitored physiological parameter of a patient) or other data (e.g., a set point from another controller). Additionally, the controllers 32, 34, 36, and 38 may each include an output circuit configured to provide signals (e.g., set point data) to a separate device or controller (e.g., 32, 34, 36, and 38). For example, the output circuit may provide signals to an actuator or a set point value to a secondary controller (e.g., 32, 34, 36, and 38). Further, each controller 32, 34, 36, and 38 may include a memory storing an algorithm configured to calculate adjustments for inducing, maintaining, and/or controlling physiological parameters of the patient 16. Such algorithms (e.g., P, PD, PI, and PID algorithms) may be utilized to safely and efficiently bring the patient's physiological parameters to a desired state. In one exemplary embodiment, a control algorithm is implemented wherein a gas or gas mixture is delivered entirely from a single source at any given time. For example, based on a monitored physiological parameter, the control algorithm may alternate the single gas source after delivery of a defined volume, time period, or breath interval. Specifically, schemes such as those used in flow-conserving supplemental oxygen delivery devices or “oxygen conservers” may be utilized, thus simplifying the delivery mechanism and utilizing the patient's lungs to mix the gases from the various single sources.

In some embodiments of the present invention, correlations between physical aspects of patients and typical patient responses to FiO2 levels may be incorporated to facilitate inducement, maintenance, and/or control of hypoxic conditions in the patients. For example, predefined proportional, integral, and/or derivative factors may be designated to facilitate tuning control loops for healthy patients, unhealthy patients, or patients with certain physical characteristics (e.g., healthy patients of a certain age or below a certain weight). In a specific example, certain integral factors for designated patient types may be used in a PI controller algorithm to make sure a certain patient SpO2 level is approached steadily. Additionally, other loop tuning factors (e.g., a derivative factor) may be utilized to improve control. In other embodiments, certain gas mixture curves may be developed to facilitate smooth blood oxygen desaturation in certain types of patients by designating gas mixture compositions and/or gas component flow rates. For example, such curves may be developed based on experiments and correlations.

As set forth above, the master controller 38 may be programmed to induce, maintain, and/or control hypoxia in the patient 16 by providing the set points and/or curves to the controllers 32, 34, and 36 such that valves 22, 26, and 28 open or close to supply an appropriate gas mixture composition (e.g., a hypoxic gas mixture). For example, the master controller 38 itself may have a steady or dynamic set point based on a physiological condition (e.g., blood saturation level) of the patient, as monitored by a sensor 46 or multiple sensors 46 that detect physiological conditions of the patient 16. For example, the master controller's set point may be a predefined estimated arterial oxygen saturation (SpO2) level in the patient 16 or a continuously changing SpO2 level. It should be noted that SaO2 is the arterial oxygen saturation of the patient 16 and SpO2 is an estimate of the SaO2, as determined via an algorithm. Thus, the master controller 38 may include a pulse oximeter used to derive SpO2 levels, or alternatively, the master controller 38 may be coupled to a separate pulse oximeter (not shown). Accordingly, the sensor 46 or sensors 46 may include a pulse oximeter sensor and/or heart rate sensor that couples to the patient 16 to detect and facilitate calculation of the patient's SpO2 (i.e., estimated blood oxygen saturation) and/or pulse. In one embodiment, the algorithm for determining the patient's SpO2 is stored in a memory of the sensor 46. Suitable sensors and pulse oximeters may include sensors and oximeters available from Nellcor Puritan Bennett Incorporated, as well as other sensor and pulse oximeter manufacturers.

A pulse oximeter and its associated sensors may be defined as a device that uses light to estimate oxygen saturation of pulsing arterial blood. For example, pulse oximeter sensors are typically placed on designated areas (e.g., a finger, toe, or ear) of the patient 16, a light is passed through designated areas the patient 16 from an emitter of the pulse oximeter sensor, and the light is detected by a light detector of the pulse oximeter sensor. In a specific example, light from a light emitting diode (LED) on the pulse oximeter sensor may be emitted into the patient's finger under control of the pulse oximeter and the light may be detected with photodetector on the opposite side of the patient's finger. Using data gained through detecting and measuring the light with the pulse oximeter sensor, a percentage of oxygen in the patient's blood and/or the patient's pulse rate may be determined by the pulse oximeter. It should be noted that values for oxygen saturation and pulse rate are generally dependent on the patient's blood flow, although other factors may affect readings.

To control the patient's SpO2 level and thus control hypoxia in the patient 16, the master controller 38 may manipulate FiO2 levels based on a comparison of one or more stored SpO2 set points and/or curves with pulse oximetry measurements of the patient's SpO2 level taken via the sensor 46. For example, if the patient's SpO2 level is above a target level, the master controller 38 may reduce FiO2 by increasing the amount of nitrogen feed (e.g., increasing flow through the nitrogen valve 30 by increasing the corresponding controller set point) while decreasing oxygen levels (e.g., decreasing flow through the oxygen and/or air valves 22 and 26 by decreasing the corresponding controller set points) in the inspiration line 12. Additionally, the master controller 38 may manipulate FiO2 levels to control heart and respiration rates that are also being monitored by the sensors 46, which may include respiration sensors. For example, if the patient's heart rate exceeds 120 BPM or if the respiration rate exceeds a set value, the master controller 38 may signal the gas supply controllers 32, 34, and 36 to increase FiO2 by increasing oxygen related set points (e.g., flow rate of air) and decreasing non-oxygen gas related set points (e.g., flow rate of nitrogen).

In one embodiment, the master controller 38 operates with the inlet portion 18 of the ventilator system 10 and the sensor 46 to maintain patient SpO2 levels down to approximately 70% by manipulating FiO2, thus controlling patient hypoxia. It should be noted that normal (e.g., during normoxic conditions) SpO2 levels for a healthy patient are approximately 97%. Maintaining SpO2 levels near 70% may reduce the patient's PaO2 from a typical value of 100 mmHg to around 37 mmHg, and create similar reductions in SvO2 and tissue O2. PaO2 may be defined as the partial pressure of oxygen in arterial blood. SvO2 or mixed venous oxygen saturation may be defined as the percentage of oxygen bound to hemoglobin in blood returning to the right side of the heart, which reflects the amount of oxygen remaining after tissues remove the oxygen they need. It should be noted that normoxia is typically maintained with FiO2 levels between 20% and 100%. Accordingly, to induce, maintain, and/or control patient hypoxia, the range of FiO2 will typically fall below 20% (e.g., an FiO2 level of 10%).

In some embodiments, it may be desirable to continually adjust the level of hypoxia (e.g., a time-varying target level or dynamic maximum safe level) rather than maintain it at a certain level. For example, for some therapeutic procedures, the goal may be to maximize hypoxia. A closed loop controller (e.g., master controller 38) may readily achieve this goal using physiological parameters. For instance, a typical response to controlled blood oxygen saturation is for the patient's heart rate to increase enough to maintain systemic oxygen transport at pre-hypoxic levels. Therefore, a closed loop controller that adjusts FiO2 to achieve a target heart rate of 120 BPM would be expected to safely achieve SpO2 values of approximately 50% in a patient whose normal resting heart rate is 60 BPM, while only allowing approximately 75% SpO2 in an out-of-shape patient with a normal resting heart rate of 90 BPM. Similarly, other closed-loop controllers may be implemented to control hypoxia while keeping multiple parameters (e.g., heart rate, blood pressure, respiration rate, tissue CO2) in safe ranges.

After being mixed according to the set points determined by master controller 38, the hypoxic or normoxic gas mixture proceeds from the inlet portion 18 of the ventilation system 10 along the inspiration line 12 to a filter/heater 48. The filter/heater 48 may operate to filter out bacteria, remove other potentially harmful or undesirable elements, and heat the gas mixture to a desired temperature. Upon exiting the filter/heater 48, the gas mixture may proceed to a flow sensor 50 (e.g., a differential pressure sensor) that measures a total flow rate of the gas mixture to the patient 16 through the inspiration line 12. Values obtained from the flow sensor 50 may be utilized in control and maintenance of patient hypoxia by providing information for use in algorithms of the master controller 38 and/or other controllers 32, 34, and 36. Eventually, the gas mixture exits the ventilation system 10 via tubing 52 for delivery to a patient via a delivery piece 54 (e.g., endotracheal tube, laryngeal mask airway, face mask, nasal pillow, and nasal canula).

Several implementations of the expiration line 14 may be utilized to handle gases (e.g., CO2 and O2) exhaled by the patient 16. For example, different exhalation sensors, filters, heaters, and configurations may be utilized dependent upon the patient's needs and/or other desirable conditions. In the embodiment illustrate by FIG. 1, gases exhaled by the patient 16 are received back into the ventilation system 10 via the expiration line 14. Once received, the exhaled gases proceed through a flow sensor 56, which measures values associated with the exhaled gases (e.g., a volumetric flow rate). Information from the flow sensor 56 may be utilized to further adjust parameters that relate to safely maintaining patient hypoxia. For example, flow rates of exhaled air from the patient may be utilized in an algorithm of the master controller 38 to compare with a predefined minimum exhalation rate for the patient. Upon exiting the flow sensor 56, the exhaled gas may proceed to a filter/heater 58, to a check valve 60, and out of the ventilation system 10. The filter heater may be adapted to cleanse the exhaled gases, and the check valve 60 may operate to prevent the exhaled gases from circulating back to the patient 16 through the ventilation system 10.

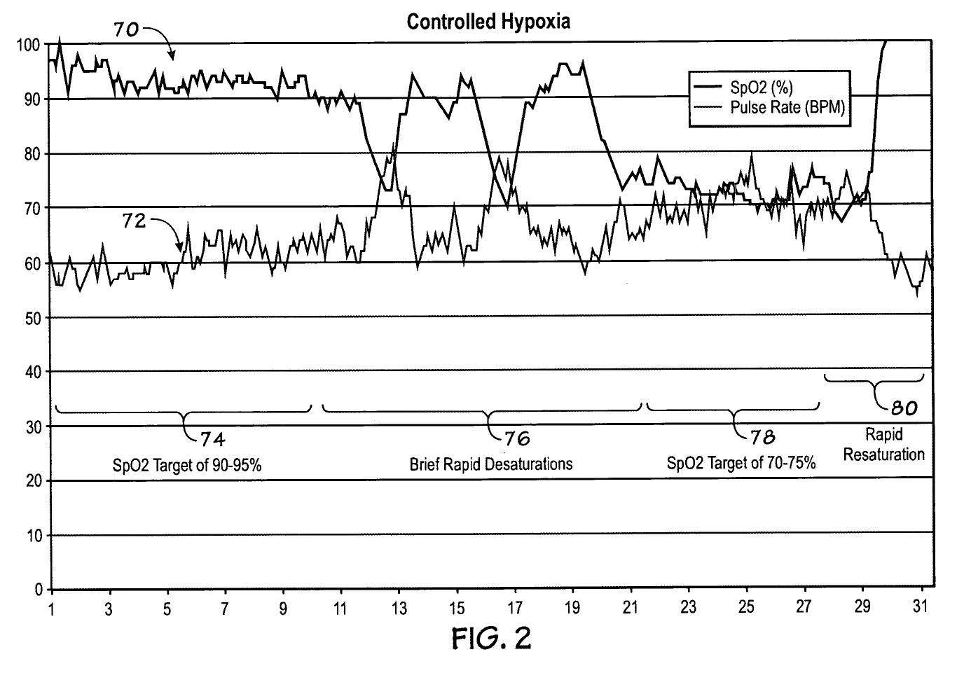

FIG. 2 is a graph illustrating data corresponding to controlled hypoxia, which may be achieved using an implementation of an exemplary embodiment of the present invention. Specifically, FIG. 2 is a graph of experimental data including a volunteer subject's SpO2 (%) and pulse rate (BPM) plotted against time (minutes). The data in FIG. 2 is representative of results that could be achieved using embodiments of the present invention to automatically control patient SpO2 levels by controlling FiO2 supplies to the patient 16. The SpO2 values are depicted by a plot line 70, and the pulse rate values are depicted by a plot line 72.

As indicated by plot line 70, the patient's SpO2 begins at a normal level (e.g., approximately 97-100%) and is maintained between 90 and 95% for a first period 74. This first period 74 in the graph illustrates an SpO2 target of 90-95%. That is, the master controller 38 of the ventilation system 10, for example, may have a set point of 90 to 95% for the patient's SpO2, which, as set forth above, causes manipulation of the gas mixture to match SpO2 levels with the set point. Next, in a middle period 76, there are brief and rapid desaturations, wherein the patient's SpO2 goes from approximately 90% to approximately 70%. Such changes in the levels of SpO2 can be automatically controlled and maintained by implementing embodiments of the present invention, wherein dynamic setpoints (e.g., time-varying target level or dynamic maximum safe level) set points are utilized or by simply changing an SpO2 set point. A third period 78 illustrates an SpO2 target of 70-75%, which may maintain hypoxia in the patient 16. Finally, a fourth period 80 illustrates rapid resaturation, wherein SpO2 levels go from approximately 70% back to normal levels. It should be noted that, as demonstrated by the plot line 72, the pulse rate of the patient increases to compensate for reduced blood oxygen.

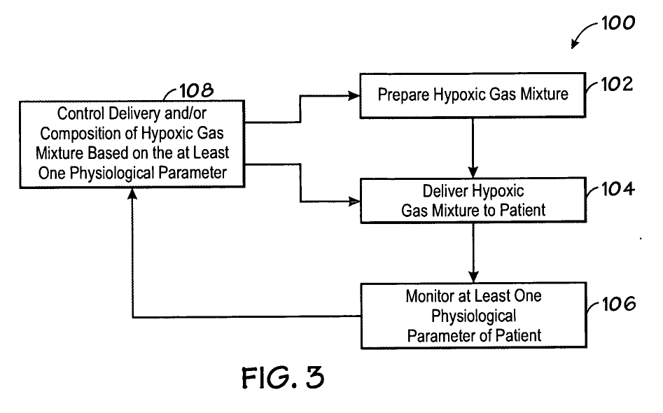

FIG. 3 is a block diagram of a method illustrating an exemplary embodiment of the present invention. The method is generally referred to by reference number 100. Specifically, method 100 begins with preparation of a hypoxic gas mixture (block 102). For example, block 102 may include mixing gases from the supplies 20, 24, and 28 in the inlet portion 18 of the ventilation system 10 to maintain a hypoxic gas mixture using the controllers 32, 34, 36, and 38, and valves 22, 26, and 30 based on data received from the sensors 46, 50, and 56. Next, block 104 represents delivering a hypoxic gas mixture to a patient, as may be achieved via the inspiration line 12 of the ventilation system 10 illustrated by FIG. 1. Further, block 106 represents monitoring at least one parameter (e.g., SpO2) of the patient, and block 108 represents controlling the delivery of the hypoxic gas mixture to the patient based on the at least one physiological parameter. For example, this can be achieved using the master controller 38 of the ventilation system 10. By continually monitoring patient physiological parameters and updating input variables, as illustrated by block 108, embodiments of the present invention may induce, maintain, and/or control patient hypoxia. In some embodiments, other procedures are also implemented to facilitate, improve, or achieve diagnostic and/or therapeutic results.

While the invention may be susceptible to various modifications and alternative forms, specific embodiments have been shown by way of example in the drawings and will be described in detail herein. However, it should be understood that the invention is not intended to be limited to the particular forms disclosed. Rather, the invention is to cover all modifications, equivalents and alternatives falling within the spirit and scope of the invention as defined by the following appended claims.

What is claimed is:

1. A method for automatically inducing, maintaining, or controlling hypoxia in a patient, comprising:
delivering a hypoxic gas mixture to the patient;
monitoring at least one physiological parameter of the patient; and
automatically controlling the delivery of the hypoxic gas mixture based on a value of the physiological parameter.

2. The method of claim 1, wherein the hypoxic gas mixture is delivered via an endotracheal tube, laryngeal mask airway, face mask, nasal pillow, nasal canula, or any combination thereof.

3. The method of claim 1, wherein the physiological parameter comprises a blood oxygenation level, a tissue carbon dioxide level, a heart rate, a blood pressure level, a respiration rate, a tissue oxygenation level, or any combination thereof.

4. The method of claim 1, comprising facilitating a diagnostic or imaging procedure for detecting regions of local hypoxia.

5. The method of claim 4, comprising detecting a tumor.

6. The method of claim 4, comprising detecting ischemic tissue.

7. The method of claim 1, comprising delivering an agent to the patient, the agent configured to localize in hypoxic tissue.

8. The method of claim 1, comprising controlling the delivery of the hypoxic gas mixture to induce, control, or maintain a therapeutic response in the patient based on a local hypoxia condition.

9. The method of claim 8, wherein the therapeutic response comprises improved patient resistance to hypoxia.

10. The method of claim 8, wherein the therapeutic response comprises neurogenesis.

11. The method of claim 8, wherein the therapeutic response comprises apoptosis.

12. The method of claim 11, comprising mediating the apoptosis by providing the patient with an agent configured to localize in hypoxic tissue.

13. The method of claim 11, comprising mediating the apoptosis by destroying local vasculature.

14. The method of claim 11, comprising mediating the apoptosis by repetitive ischemia-reperfusion injury.

15. The method of claim 1, comprising controlling delivery of the gas mixture to maintain a fixed or time-varying target level of hypoxia.

16. The method of claim 1, comprising controlling delivery or content of the gas mixture to maximize the hypoxia within predefined parameters.

17. A ventilation system for automatically inducing, maintaining, or controlling hypoxia in a patient, comprising:
a delivery mechanism configured to deliver a hypoxic gas mixture to the patient; and
a controller configured to monitor at least one physiological parameter of the patient and to automatically adjust delivery of the hypoxic gas mixture based on a comparison of a value of the monitored physiological parameter with a stored physiological parameter.

18. The system of claim 17, wherein the delivery mechanism includes an endotracheal tube, laryngeal mask airway, face mask, nasal pillow, nasal canula, or any combination thereof.

19. The system of claim 17, wherein the delivery mechanism includes at least one gas supply tank and a control valve configured to provide designated amounts of gas from the gas supply tank to the patient.

20. The system of claim 17, comprising at least one sensor configured to determine the at least one physiological parameter of the patient.

21. The system of claim 17, wherein the controller comprises a pulse oximeter monitor and sensor.

22. A controller, comprising:
an input circuit configured to receive data relating to at least one physiological parameter of a patient;
a memory storing an algorithm configured to calculate adjustments for a set point for delivery of a hypoxic gas mixture to the patient based on a comparison of the data relating to the at least one physiological parameter with a master set point for the at least one physiological parameter; and
an output circuit configured to send the set point to a delivery mechanism, the delivery mechanism being configured to deliver the hypoxic gas mixture to the patient.

23. The controller of claim 22, comprising a plurality of flow controllers configured to supply a designated gas mixture.

24. The controller of claim 22, comprising a target entry circuit configured to receive the master set point.

25. A method of manufacturing a ventilation system for automatically inducing, maintaining, or controlling hypoxia in a patient, comprising:
providing a delivery mechanism configured to deliver a hypoxic gas mixture to the patient; and
providing a controller configured to monitor at least one physiological parameter of the patient and to automatically adjust delivery of the hypoxic gas mixture based on a comparison of the monitored physiological parameter with a stored physiological parameter.

26. The method of claim 25, comprising providing an input circuit in the controller, the input circuit configured to receive data relating to the at least one physiological parameter of the patient.

27. The method of claim 25, comprising providing a memory in the ventilation system, the memory storing an algorithm configured to calculate adjustments for a delivery set point for delivery of the hypoxic gas mixture to the patient based on the comparison of the value of the physiological parameter with the stored physiological parameter.

28. The method of claim 25, comprising providing an output circuit in the ventilation system, the output circuit configured to send the delivery set point to the delivery mechanism.
